(19) United States
(12) Patent Application Publication
(10) Pub. No.: US 2004/0106818 A1
(43) Pub. Date: Jun. 3, 2004

(54) Process for the preparation of cyclohexanol derivatives

(76) Inventors: Lan Zhiyin, Chongqing (CN); Shi Kaiyun, Chongqing (CN); Mo Qizhuang, Chongqing (CN); Li Yulin, Lanzhou (CN)

Correspondence Address: LATHROP & GAGE LC, 4845 PEARL EAST CIRCLE, SUITE 300, BOULDER, CO, 80301, US

(21) Appl. No.: 10/638,845

(22) Filed: Aug. 11, 2003

(30) Foreign Application Priority Data
Nov. 29, 2002 (CN) CN02153015.7

(51) Int. Cl. C07C 253/30; C07C 231/12

(52) U.S. Cl. 558/369; 564/171

(57) ABSTRACT

A reaction of a para-substituted aryl composition with cyclohexanone is facilitated by a metal hydride, such as NaH, KH, LiH, MgH₂, CaH₂, AlH₃, and/or LiAlH₄ to make first intermediates useful in producing a drug commonly known as Venlafaxine. Alternatively, lithium diisopropylamide (diisopropylamino lithium) may be used in place of the metal hydride. The first intermediates may be further reacted to form second intermediates in a reduction that is facilitated by Rainey nickel or a metal hydride. These reaction processes may each occur in an organic solvent, which delivers highly pure reaction products in high yield.

### RELATED APPLICATIONS

This application claims benefit of priority to application Ser. No. 02153015.7 filed Nov. 29, 2002 in the People's Republic of China and is a continuation-in-part of that application.

### BACKGROUND OF THE INVENTION

A number of nontricyclic antidepressants have recently been developed to treat depression and anti-social disorders. One of these compounds, Venlafaxine, chemically named (+)-1-[2-(dimethylamino)- 1-(4-methoxyphenyl)ethyl]-cyclohexanol, is widely used. The manufacturing cost of Venlafaxine is relatively high.

Literature reports including EP 01 12669B and U.S. Pat. No. 4,535,186 describe the preparation of cyclohexanol derivatives as intermediates for making Venlafaxine. A variety of α-aryl-α-(1-hydroxylcyclohexyl) ethylamine derivatives are synthesized by reacting α-aryl acetonitriles or α-aryl-N,N-dimethyl acetamides with cyclohexanone. The reactions are performed using organo-lithium metal compounds of nitriles or amides, which are reacted with cyclohexanone using tetrahydrofuran as solvent at a low temperature below −50° C. The yield is reported to be less than 50%. It is costly and difficult to run large scale commercial production at reaction temperatures below −50° C.

U.S. Pat. No. 5,043,466 describes another process of preparing α-aryl-α-(1-hydroxylcyclohexyl) ethylamine derivatives. In that process, α-aryl-α-(1-hydroxylcyclohexyl) acetonitriles or α-aryl-N,N-dimethyl (1-hydroxylcyclohexyl) acetamides are reacted with organic alkali metal compounds, such as lithium, sodium, potassium, or magnesium halide, to form a carbanion in a mixed solvent including 80-100% (w/w) of one or more hydrocarbons and 0-20% (w/w) of one or more ethers at −40° C. to about 40° C. This organo metallic complex is then reacted with cyclohexanone to prepare α-aryl-α-(1-hydroxylcyclohexyl) ethylamine derivatives. The use of n-butyllithium is a great inconvenience in large-scale operation because butyllithium is a highly hazardous chemical.

A more recent patent, U.S. Pat. No. 6,504,044, reports another method for preparing 1-[cyano(aryl)methyl] cyclohexanol by reacting cyclohexanone with aryl acetonitrile in the presence of sodium hydroxide or potassium hydroxide. Although the low reaction temperature has been avoided, the use of strong bases such as sodium hydroxide renders the procedure more complicated and costly in large scale production. Moreover, the use of relatively expensive phase transfer catalysts such as n-butyl ammonium iodide adds significantly to the cost of production. In the absence of phase transfer catalysts, the yield of the process disclosed in U.S. Pat. No. 6,504,044 is significantly reduced.

There remains a need to provide more economical processes for preparing Venlafaxine intermediates which do not require facilitation by phase transfer catalysts and can be carried out under milder conditions more suitable for large scale production.

### SUMMARY

The present disclosure advances the art and overcomes the problems outlined above by providing a process of making intermediates for nontricyclic antidepressants, such as Venlafaxine and the like. The process is advantageously capable of avoiding low process temperatures, as well as avoiding use of phase transfer catalysts and butyllithium in the manufacture of Venlafaxine and its derivatives.

Venlafaxine intermediates of interest include cyclohexanol derivatives, such as N,N-dimethyl-2-(1-hydroxylcyclohexyl)-2-(4-hydroxylphenyl) ethylamine, or N,N-dimethyl-2-(1-hydroxylcyclohexyl)-2-(4- methoxylphenyl) ethylamine, and salts thereof. Once obtained, these intermediates may be used to manufacture Venlafaxine, for example, as described in U.S. Pat. No. 5,043,466. In one embodiment, a process of making the intermediates includes reacting a para-substituted aryl compound with cyclohexanone in the presence of an alkali metal hydride or other organic compound to form a bridged cyclohexyl-phenyl composition according to Equation (1) below:

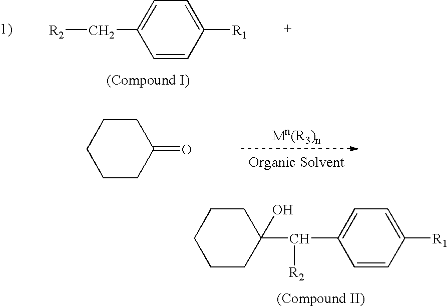

wherein R₁ is OH or OCH₃; R₂ is CN, CONH₂, CONHCH₃ or CON(CH₃)₂; R₃ is H; M denotes an alkali metal or complex metal, e.g., M₁M₂ where M₁ and M₂ are different metals; and n is the net oxidation state of M. When M includes lithium, R₃ may also be an organic ligand, such as an amine having a carbon number from 2 to 6, and the use of lithium diisopropylamide (diisopropylamino lithium) is particularly preferred.

The reaction according to Equation (1) is performed using organic solvents and appropriate reaction conditions, taking care to minimize or eliminate water from the reaction solution because water degrades the compound Mⁿ(R₃)ₙ. Materials suitable for use as Mⁿ(R₃)ₙ include, for example, NaH, KH, LiH, MgH₂, CaH₂, AlH₃, and LiAlH₄. The reaction temperature ranges, in a preferred sense, from −10° C. to 30° C. The organic solvents may include commercially available solvents, such as alkanes and aryl compositions. By way of example, n-hexane, toluene, or a mixture thereof are suitable for use as the organic solvents.

Compound II may be further reduced to form other intermediates, as shown below in Equations (2) and (3), respectively

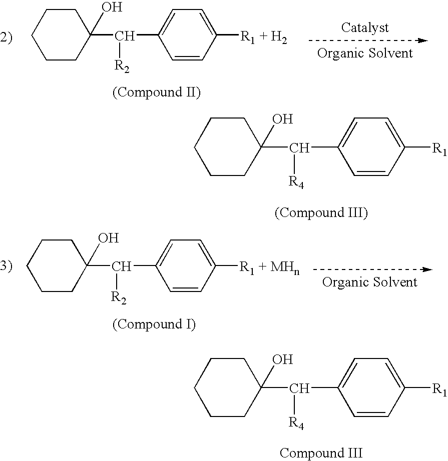

wherein R₁ is OH or OCH₃; R₂ is CN, CONH₂, CONHCH₃ or CON(CH₃)₂; and R₄ denotes CH₂NH₂. M and n are defined above. In context of Equation (3), for example, MHₙ is preferably AlH₃ and/or LiAlH₄.

The reactions according to Equations (2) and (3) are both performed using organic solvents under appropriate reaction conditions to reduce Compound II. In the reaction according to Equation (2), Raney nickel is used as a catalyst, while the reaction according to Equation (3) is performed using a hydride including one or more elements from Groups 1A and 3A of the Periodic table as a reducing agent, for example, aluminum hydride or lithium aluminum hydride as a reducing agent.

The foregoing reactions are prefereably but optionally conducted at a temperature ranging from −10° C. to 30° C., and more prefereably from −10° C. to 10° C.

Where diisopropylamino lithium is used in Equation (1), the reaction temperature is suitably from −10° C. to 50° C., and the diisopropylamino lithium solution may be obtained by reacting metalic lithium with phenethylene. In the case of a lithium reagent, the organic solvent may be, for example, a mixture of ether and aromatic hydrocarbon, such as toluene or styrene. The ether may be, for example, diethyl ether, diisopropyl ether, ethylene glycol dimethyl ether, and tetrahydrofuran.

Solvents for use according to Equations (2) and (3) may be an alcohol or a mixture of different alcohols. Reaction temperatures for these reduction reactions may range, for example, from 0° C. to 40° C. Catalytic hydrogenation facilitated by Rainey nickel may be done under atmospheric pressure. AlH₃ used in Equation (3) may be obtained by reacting lithium aluminum hydride (LiAlH₄) with anhydrous aluminum chloride in an ether solvent.

### DETAILED DESCRIPTION

There will now be shown, by way of nonlimiting examples, a process of making Compound II, as shown in Equation (1) above. The process uses readily available organic solvents and an alkali metal hydride or an organic alkali metal compound such as organo-lithium to prepare Compound II. The two methods to prepare Compound II are discussed below. The general concepts and instrumentalities are illustrated by detailed examples, which embody specific implementation of the general concepts and instrumentalities and should not be construed as setting a limit to the scope of the processes.

The reaction between Compound I and cyclohexanone according to Equation (1) occurs in the presence of a base, such as NaH, KH, LiH, MgH₂, CaH₂, AlH₃, and LiAlH₄ at a temperature ranging from −10° C. to 30° C. The process utilizes commercially available solvents, for example, n-hexane and toluene. The reaction advantageously occurs over a short reaction period, produces high yields (˜80%) and incurs lower raw material costs, when compared with prior methods. Compound II may be isolated in high yield by precipitation from the organic solvent.

When the composition Mⁿ(R₃)ₙ is an organolithium, the organic solvent provides a reductive solvent atmosphere that is well suited for the reaction between Compound I and cyclohexanone in which phenylethylene, metal lithium and diisopropylamine are used to form a lithium diisopropylamine solution in ethylbenzene, Compound I and cyclohexanone is thereafter added to produce Compound II.

The reactions according to Equations (2) and (3) provide two alternative processes for the preparation of intermediates having the formula of Compound III by reduction of Compound II, for example, using Raney Nickel or aluminum hydride. These two methods are described below.

Equation (2) shows the use of a Raney nickel catalyst and supplemental hydrogen to reduce Compound II to Compound III in an alcohol based solvent environment at a temperature ranging, in a preferred sense, from about 0° C. to 30° C.

Equation (3) shows reduction of Compound II using aluminum hydride to form Compound III in a mixed solvent system. AlH₃ is prepared by the reaction of anhydrous aluminum chloride with lithium aluminum hydride in a mixed solvent system at room temperature.

A process is also described for the preparation of an acid salt of Compound III. Venlafaxine can be produced from either Compound III or the acid salt of Compound III.

### EXAMPLE 1

### Preparation of 1-[Cyano(p-methoxyphenyl)methyl]cyclohexanol (Compound II)

### Method I—Use of Sodium Hydride

A reaction mixture was prepared by sequentially combining 10 mL of n-hexane, 10 mL of toluene and 710 mg of sodium hydride (60%, 17.8 mmol) in a dry flask. The mixture was stirred for 15 minutes at room temperature. A 2.5 g (17.0 mmol) quantity of p-methoxyphenylacetonitrile was slowly added to the reaction mixture over a 10 minute interval. The reaction mixture was stirred for 50 minutes at room temperature.

The reaction mixture was cooled to −5° C., a 2.18 g (22.0 mmol) of cyclohexanone was added dropwise over 10 minutes. The reaction mixture was stirred for 5 hours to the reaction end point, which was monitored by thin layer chromatography (TLC) using a solvent of ligroin: ethyl acetate=4:1. Reaction was deemed complete when only one spot was detected using TLC. At completion of the reaction, a quantity of 5% diluted hydrochloric acid was added dropwise to adjust pH to 6-7, and the mixture was stirred for 15 minutes. Precipitate was filtered under vacuum, and the filter cake was washed with water. The filter cake was dried to obtain a crude sample.

The crude sample was purified by recrystallization using a volume of toluene in mililiters equal to three times the crude product weight in grams. A 3.33 g quantity of the purified product, 1-[Cyano(p-methoxyphenyl) methyl] cyclohexanol (Compound II) was obtained, representing an 80% yield. Composition of the reaction product was initially confirmed by melting point, which was assessed at mp. 124-125° C. A melting point of 123-126° C. for this product is published in the literature.

Composition of the final reaction product was further confirmed using infrared spectroscopy (IR), nuclear magnetic resonance (NMR) and mass spectroscopy (MS), which produced these results:

IR: 3494 cm⁻¹ (v O—H), 2935 cm⁻¹ (v O—H), 2240 cm⁻¹ (v CN), 1612 cm⁻¹ ( v Aromatic ring), 1512 cm⁻¹ (v Aromatic ring);

¹H-NMR(CDCL₃, TMS): δ 7.26 ppm (d, 2 H, Ar H), δ 6.89 ppm (d, 2 H, Ar H), 3.8 ppm(s,3 H, OCH₃),3.73 ppm (s, 1 H, CHCN), 1.73-1.17 ppm (m, 10 H, Cyclohexyl H), (¹H-NMR(CDCL₃): δ 7.32 ppm and 6.95 ppm (q, 4 H, Ar H), 3.8 ppm(s, 3 H, OCH₃), 3.76 ppm (s, 1 H, CHCN), 1.56 ppm(m, 10 H, Cyclohexyl H in Literature);

MS: m/z 245 (M+; EIMS)( CIMS m/e=M⁺+1=246 in Literature).

### EXAMPLE 2

### Preparation of 1-[Cyano(p-methoxyphenyl)methyl]cyclohexanol (Compound II)

### Method II—Use of Diisopropylamino Lithium

A reaction mixture was prepared by sequentially mixing 2500 mL of absolute ether, 1250 mL of diisopropylamine and 50 g of metal lithium (in plate form) into a 5000 mL three-necked round-bottomed flask under dry nitrogen. The reaction mixture was heated for reflux using a water-bath. A mixture of 464 mL of phenylethylene dissolved in absolute ether was added dropwise to the reaction mixture, which was refluxed for 2 hours. The lithium plates completely reacted to form a gray solution.

The reaction mixture was cooled below 5° C. A solution including 680 mL of p-methoxyphenylacetonitrile (5 mol, diluted with 400 mL of toluene) and 550 mL of cyclohexanone (5.3 mol, diluted with 300 mL of toluene) was added dropwise to the reaction mixture. A green-yellowish solution was obtained. The solution was poured into a mixture of 1 kg of ice and 600 mL of concentrated hydrochloric acid to form a precipitate. The precipitate was isolated by filtration to yield 610 g of crude product. The filtrate was separated by phase, and the organic layer was concentrated by vacuum to obtain an additional other 190 g of the crude product. The total quantity of crude product was 800 g, for a yield of 65%. The crude product was purified using toluene recrystallization, as in Example 1, to obtain 600 g of final product, 1-[Cyano(p-methoxyphenyl)methyl] cyclohexanol (Compound II). Purity was confirmed by melting point, which was determined as mp. 124-126° C., as well as IR, NMR, and MS.

### EXAMPLE 3

### Preparation of 1-[2-Amino-1-(p-methoxyphenyl)ethyl]cyclohexanol (Compound III) and Its Salts

### Method 1—Use of Rainey Nickel

A 2.45 g(10 mmol) quantity of the 1-[cyano(p-methoxyphenyl)methyl] cyclohexanol obtained in Example 1 and 1.0 g of catalyst Raney Nickel were dispersed to a 50 mL flask containing 30 mL of methanol. Air was purged from the flask four times with hydrogen. The mixture was stirred vigorously at room temperature under a blanket of hydrogen for about 15 hours to complete the reaction. The reaction progress was periodically monitored using TLC, and was deemed complete when no more hydrogen was being absorbed and only one spot was detected on the TLC plate.

After completion of the reaction, stirring stopped and the reaction mixture stood. A precipitate formed. The reaction mixture was filtered to remove the precipitate as a filter cake. The Rainey nickel was removed from the filter cake. The filter cake was then washed 3 times with methanol. The methanol wash was combined wit the supernatant from the reaction mixture. Methanol was removed by vacuum distillation to leave 2.50 g of an oily crude reaction product, which was purified using silia gel column chromatography (CH₂Cl₂:MeOH=30:1 v/v). A 2.06 g quantity of 1-[2-Amino-1-(p-methoxyphenyl)ethyl] cyclohexanol was obtained to provide an 83% yield. TLC analysis of the final product using as a solvent CH₂Cl₂:MeOH=30:10 v/v produced one primary spot, confirming the purity of the reaction product. This intermediate can be directly used for preparing Venlafaxine and its salts.

### EXAMPLE 4

### Preparation of 1-[2-Amino-1-(p-methoxyphenyl)ethyl]cyclohexanol (Compound III) and Its Salts

### Method 2—Use of Lithium Aluminum Hydride

A 100 g quantity of LiAlH₄ and 1500 mL of absolute ether were added into a 500 mL flask to prepare a reaction mixture at room temperature. A solution was prepared with 125 g of anhydrous aluminum trichloride (AlCl₃) into 600˜1000 mL of absolute ether under nitrogen, and added dropwise to the reaction mixture. The reaction mixture was cooled in an ice-water bath. A solution containing 314 g of 1-[Cyano(p-methoxyphenyl)methyl] cyclohexanol obtained in Example 1 dissolved in 800 mL of tetrahydrofuran (THF) was added dropwise to the reaction mixture. The reaction mixture thus formed was allowed to react for 5 hours at room temperature. A 400 mL quantity of THF:H₂O(1:1) was added, followed by 400˜600 mL of 50% saturated sodium hydroxide (NaOH) solution until the resulting solid-liquid had clearly separated and the solid appeared white. The liquid organic layer was decanted, and the remaining precipitate was washed with ether. The remaining precipitate was combined with the decanted organic layer and concentrated by vacuum to obtain an oily product which was 1-[2-Amino-1-(p-methoxyphenyl)ethyl] cyclohexanol. TLC analysis produced a single spot, ninhydrin positive, using a solvent of chloroform-methanol-acetic acid (80:10:10 v/v). This oily product can be directly used for preparing Venlafaxine HCl.

### EXAMPLE 5

### Preparation of 1-[2-Amino-1-(p-methoxyphenyl)ethyl] cyclohexanol (Compound III) and Its Salts

### Method 3—Preparation of 1-[2-Amino-1-(p-methoxyphenyl)ethyl] cyclohexanol (Compound III) Hydrochloride

A 1.0 g of the crude product obtained in Example 4 was dissolved in 2 mL of methanol at room temperature in a 10 mL conical flask. The mixture was cooled in an ice-water bath and stirred. Hydrogen chloride gas was added to the reaction flask until the gas was no longer introduced under stirring. Ether was added dropwise until some slight turbidity was observed. Stirring ceased, and the mixture stood for 10 hours. 860 mg of white solid is precipitated. Initial purity and composition were confirmed by melting point, which was determined to be m. p.=168-170° C. (168-172° C. is reported in the Literature). Composition was confirmed to be Compound III hydrochloride by IR and NMR:

IR: 3364 cm⁻¹ ( v N—H), 2933 cm⁻¹ (v O—H), 1611 cm⁻¹ (v Aromatic ring), 1512 cm⁻¹ ( v Aromatic ring).

¹H-NMR(CDCL₃, TMS): δ 7.09 ppm (d, 2 H, Ar H), δ 6.76 ppm (d, 2 H, Ar H), 3.70 ppm (s, 3 H, OCH₃), 3.16 ppm (dd, 1 H, CH), 3.04 ppm (dd, 1 H, CH), 2.57 ppm (m, 3 H, CH and NH₂), (m, 10 H, Cyclohexyl H), (¹H-NMR (DMSO-d6 in Literature): δ 7.85 ppm (s, 3 H, NH₃⁺), 3.75 ppm (s, 3 H, OCH₃), 3.20 ppm (m, 3 H, CHCH₂), 1.35 ppm (m, 10 H, Cyclohexyl H).

1. A process for preparing compounds of the formula 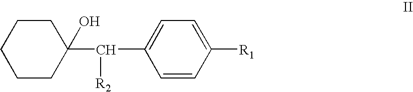
wherein R₁ denotes OH, or OCH₃, and R₂ denotes CN, CONH₂, CONHCH₃ or CON(CH₃)₂
by reacting nitriles or amides of the formula 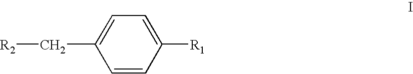
wherein R₁ denotes OH, or OCH₃, and R₂ denotes CN, CONH₂, CONHCH₃ or CON(CH₃)₂,
with cyclohexanone in the presence of a solvent and a base selected from the group consisting of NaH, KH, LiH, MgH₂, CaH₂, AlH₃, LiAlH₄ and combinations thereof.

2. The process of claim 1, wherein the step of reacting nitriles or amids occurs at a temperature in a range from −10° C. to 30° C.

3. The process of claim 1, wherein the reaction temperature ranges from −10° C. to 10° C.

4. The process of claim 1, wherein the base is sodium hydride.

5. The process of claim 1, wherein the solvent includes an alkane, and aromatic hydrocarbon, or a mixture of alkane and aromatic hydrocarbon.

6. A process for preparing compounds of the formula 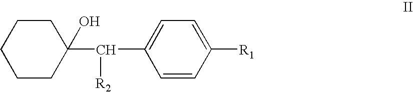
wherein R₁ denotes OH, or OCH₃, and R₂ denotes CN, CONH₂, CONHCH₃ or CON(CH₃)₂
by reacting nitrites or amides of the formula 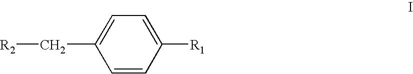
wherein R₁ denotes OH, or OCH₃, and R₂ denotes CN, CONH₂, CONHCH₃ or CON(CH₃)₂,
with diisopropylamino lithium solution in a solvent.

7. The process of claim 6, wherein the step of reacting nitrites or amides occurs at a temperature ranging from −10° C. to 50° C.

8. The process of claim 7, wherein the temperature ranges from −10° C. to 10° C.

9. The process of claim 6, wherein the diisopropylamino lithium solution is obtained by reacting metalic lithium with diisopropylamine.

10. The process of claim 6, wherein the solvent is a mixture of ether and aromatic hydrocarbon.

11. The process of claim 10, wherein the ether is selected from the group consisting of diethyl ether, diisopropyl ether, ethylene glycol dimethyl ether, and tetrahydrofuran.

12. A process for preparing compounds of the formula 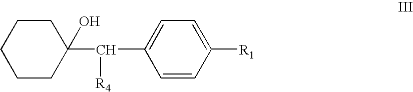
wherein R₁ denotes OH, or OCH₃, and R₄ denotes CH₂NH₂, by reducing, in a solvent, a compound of the formula 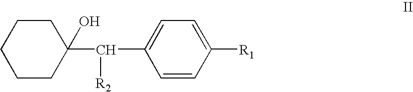
wherein R₁ denotes OH, or OCH₃, and R₂ denotes CN, by catalytic hydrogenation.

13. The process of claim 12, wherein the step of reducing the compound occurs under atmospheric pressure.

14. The process of claim 12, wherein the solvent is an alcohol or a mixture of different alcohols.

15. The process of claim 12, wherein the step of reducing the compound occurs temperature ranging from 0° C. to 40° C.

16. The process of claim 12, wherein the catalytic hydrogenation is carried out under atmospheric pressure.

17. The process of claim 12, wherein the catalytic hydrogenation is facilitated using Raney Nickel.

18. A process for preparing compounds of the formula 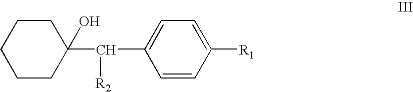
wherein R₁ denotes OH, or OCH₃, and R₄ denotes CH₂NH₂, by reducing a compound of the formula 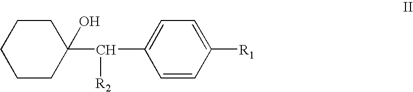
wherein R₁ denotes OH, or OCH₃, and R₂ denotes CN, with a metal hydride in a solvent.

19. The process of claim 18, wherein the step of reducing the compound occurs at a temperature ranging from −10° C. to 30° C.

20. The process of claim 19, wherein the temperature ranges from −10° C. to 10° C.

21. The process of claim 18, further including a step of producing the metal hydride as AlH₃ obtained by reacting lithium aluminum hydride (LiAlH₄) with anhydrous aluminum chloride in an ether solvent.

22. The process of claim 18, wherein the solvent is an ether selected from the group consisting of diethyl ether, diisopropyl ether, and tetrahydrofuran.
